(19) United States
(12) Patent Application Publication
(10) Pub. No.: US 2016/0270701 A1
(43) Pub. Date: Sep. 22, 2016

(54) REMOTE USER INTERFACE WITH EAR SELECTION SWITCH

(71) Applicant: GN Otometrics A/S, Taastrup (DK)

(72) Inventor: Johannes LANTZ, Malmo (SE)

(73) Assignee: GN Otometrics A/S, Taastrup (DK)

(21) Appl. No.: 15/069,750

(22) Filed: Mar. 14, 2016

(30) Foreign Application Priority Data
Mar. 19, 2015 (DK) PA 2015 70157

(51) Int. Cl. A61B 5/12

(52) U.S. Cl. CPC A61B 5/121

(57) ABSTRACT

A remote user interface is provided for connection with an audiologic test apparatus for audiologic testing and comprising a remote user interface housing accommodating a mechanically non-latching switch for selection of one of the right ear or left ear of a patient for testing with the audiologic test apparatus.

### RELATED APPLICATION DATA

This application claims priority to and the benefit of Danish Patent Application No. PA 2015 70157 filed on Mar. 19, 2015, pending. The entire disclosure of the above application is expressly incorporated by reference herein.

### FIELD

A new remote user interface for an audiologic test apparatus, such as a middle ear analyser, is provided.

### BACKGROUND

In order to perform audiologic tests, such as tympanometry, ipsi and contra acoustic reflex measurements, otoacoustic emission measurements, pure tone audiometry, etc., an ear probe is provided for positioning in the ear canal, typically, for provision of a selected air pressure in the ear canal and/or a selected pure tone or other sound and/or for measurement of the sound pressure level in the ear canal. The audiologic tests are controlled by an audiologic test apparatus connected with the ear probe and the measurement results are recorded by the audiologic test apparatus. A hand-held ear probe may be used for screening purposes, while an ear probe that is retained more securely in or at the ear canal of a human is typically preferred for clinical tests.

### SUMMARY

When clinical or screening tests are performed, there is a need for recording which of the left ear and right ear that is subjected to the test in question.

For this purpose, a switch is conventionally provided at the audiologic test apparatus for selection of which of the left ear and the right ear that is subjected to a test. For example, a reflex test may be performed, wherein the left ear is selected to be tested and the selection may be displayed on a screen of the audiologic test apparatus as “Reflex: Left”.

However, it is cumbersome and time consuming for the operator of an audiologic test apparatus with an ear probe to have to move away from the patient in order to select which ear that is going to be tested and in order to select the other ear when testing of the first ear has been performed. Time is wasted and communication with the patient may be interrupted and the calm atmosphere that is desired during audiologic testing may be disturbed.

In order to eliminate the need for the operator of the audiologic apparatus and the ear probe, to move proximate the audiologic test apparatus during testing, a new remote user interface is provided that is connected with the audiologic test apparatus and that has a remote user interface housing accommodating a switch for selection of the right ear or the left ear of the patient for testing with the ear probe. During use, the remote user interface housing is positioned proximate the patient.

The remote user interface housing is preferably configured for mechanical connection with the ear probe of the audiologic test apparatus in such a way that the remote user interface will be located proximate the patient during the audiologic testing of the patient. With the remote user interface located proximate the patient, e.g. worn by the patient, the operator of the ear probe may perform selection of the ear to be subjected to the test, or being subjected to the test, without being forced to move proximate to the audiologic test apparatus connected with the ear probe for the purpose of ear selection.

For example, an operator of an audiologic test apparatus with an ear probe performing audiologic testing of a patient's ears may recognize, after having positioned the ear probe appropriately in the desired ear of the patient that he or she has not selected the correct ear for testing as required to be able to record the test results together with identification of the selected ear, i.e. correct information on whether the test results relate to the right ear or the left ear. Conventionally, the operator in this situation had to move away from the patient to the audiologic test apparatus to perform the selection of the right ear or left ear and revert to the patient and possibly check or even re-establish the desired positioning of the ear probe. However, the operator desires to maintain focus on the patient and the patient's reactions and wants to minimize disturbing activities, such as moving away from the patient. This is avoided with the new remote user interface.

Preferably, the switch for selection of the right ear or the left ear for testing with the ear probe does not provide other functions in response to its actuation, for example the switch is not used for simultaneously control of the start and stop of the audiologic test.

Preferably, the switch is a momentary switch also called a mechanically non-latching switch, i.e. the switch reverts to the same mechanical state or position upon activation as for example a spring biased push button switch. The mechanically non-latching switch may be a touch sensitive switch that is actuated by touching its surface with a finger and that provides a switch signal when actuated, e.g. by making contact between two poles of the switch or by breaking contact between two poles of the switch, or in any other suitable way well-known in the art of touch sensitive switches.

The touch sensitive switch may be positioned so that a surface of the touch sensitive switch is flush with a surface of the remote user interface housing.

The touch sensitive switch may have a surface that forms a part of a surface of the remote interface housing.

The mechanically non-latching switch may be a push button switch, e.g. biased with a spring, e.g. with appropriate tactile feedback, when the switch is actuated, i.e. depressed or released, and that provides a switch signal when actuated, e.g. by making contact between two poles of the switch or by breaking contact between two poles of the switch, or in any other suitable way well-known in the art of push buttons.

A mechanically latching switch has a moving part called the actuator with two or more mechanical states or positions and changes mechanical state or position upon actuation. The switch remains in its changed state or position until the next actuation is performed. For example, the well-known toggle switch or flip switch, e.g. a wall switch, has two mechanical states or positions, and a user actuates the switch by changing its position for example to turn on a lamp in one position and turn the lamp off in another position.

The mechanically non-latching switch may be configured for alternating selection of the right ear or the left ear for testing with the audiologic test apparatus by repeated actuation of the switch.

The remote user interface housing may accommodate two switches of the same or different ones of the above-mentioned types of mechanically non-latching switches, each of which is provided for selection of the right ear or the left ear, respectively.

The remote user interface housing may accommodate one or two switches of the above-mentioned types of mechanically non-latching switches configured for controlling start and stop of testing with the audiologic apparatus as is well-known in the art.

The remote user interface housing may accommodate an indicator of the one of the right ear and left ear that is currently selected for testing with the ear probe.

The indicator may provide visual indication of the selected ear, e.g. the indicator may comprise a light source, such as a lamp, a LED, etc., that turns on when one of the right ear and left ear is selected for testing and that turns off when the other one of the right ear and left ear is selected for testing.

The indicator may comprise a light source, such as a lamp, a LED, etc., that emits light of a certain colour when a respective one of the right ear and left ear is selected for testing and that emits light of a different colour when the other one of the right ear and left ear is selected for testing. For example, the light source may turn orange when the right ear is selected for testing and the light source may turn blue when the left ear is selected for testing. Alternatively or additionally, the light source may blink in a certain sequence to indicate selection of a respective left or right ear.

The light source may be integrated with the mechanically non-latching switch.

Instead, or additionally, the indicator may provide acoustic indication of the selected ear, e.g. the indicator may comprise a sound source, such as a miniature loudspeaker, that emits a specific sound when a respective one of the right ear and left ear is selected for testing and that emits a different sound when the other one of the right ear and left ear is selected for testing. The two sounds may be pure tones of different frequencies, or short different melodies, or different sequences of the same sound, e.g. one beep when the right ear is selected for testing and two beeps when the left ear is selected for testing.

The indicator may also be configured to indicate when a measurement is in progress, and/or air leakage and/or instability in the measured ear cavity volume, etc.

Instead, or additionally, indication of any of the above conditions indicated by the indicator of the remote user interface may be provided by the audiologic test apparatus, e.g. by respective indications displayed on a screen of the audiologic test apparatus, e.g. in well-known ways.

Instead, or additionally, indication of any of the above conditions indicated by the indicator of the remote user interface may be provided by the ear probe.

The remote user interface housing may be intended to rest at the patient's body, e.g. the remote user interface may be connected to the audiologic test apparatus with a cable that may rest together with the remote user interface housing across a shoulder of the patient.

The mechanically non-latching switch for selection of the right ear or the left ear of the patient for testing with the audiologic test apparatus may be duplicated, i.e. a housing of the audiologic test apparatus different from the remote user interface housing may also accommodate a user interface for selection of the right ear or the left ear of the patient for testing with the audiologic test apparatus so that an operator of the audiologic test apparatus has the option of selecting the left ear or the right ear for testing or for studying recorded test results relating to the selected ear either with the remote user interface or with the user interface of the audiologic test apparatus.

The indicator may also be duplicated, i.e. the housing of the audiologic test apparatus may also accommodate an indicator, e.g. of the types mentioned above with relation to the remote user interface, so that the ear currently selected may be indicated both at the housing of the audiologic test apparatus and at the remote user interface housing.

The audiologic test apparatus may be a self-contained unit with its own user interface with switches and a display, possibly a keyboard, as is well-known in the art of audiologic test apparatuses.

The audiologic test apparatus may also be based on a general purpose computer, such as a PC, laptop, PDA, smartphone, etc., for recording to the test results and that comprises the user interface of the audiologic test apparatus and that is interconnected with and controls a special purpose test apparatus with the required measurement circuitry and devices for performing the audiologic tests.

The recorded test results may be stored together with the identification of the right ear or the left ear that the stored test results relate to in the audiologic test apparatus and possibly other relevant information, such as the date, time, patient ID, operator ID, etc.

The recorded data may be transferred to another device, such as a remote storage, such as a server interconnected with the audiologic test apparatus through a Wide Area Network, such as the Internet.

The ear probe may be connected with a cable to the audiologic test apparatus in a well-known way.

The cable may include electrical conductors for interconnection of a microphone and a loudspeaker and switches and possible indicators, respectively, accommodated in the probe housing.

The cable may also have an air conduit for interconnection with a pump in the audiologic test apparatus for control of the air pressure in the ear canal subjected to clinical testing or screening in a way well-known in the art of audiologic testing.

Likewise, the ear probe may be connected to the remote user interface housing with a cable and the remote user interface housing may be connected to the audiologic test apparatus with a cable so that the ear probe may be connected with the audiologic test apparatus via the remote user interface housing.

The probe housing may constitute the remote user interface housing.

The ear probe may for example be used for acoustic impedance and static pressure measurements in the ear canal. These measurements, possibly in combination with audiometry, such as pure tone audiometry or speech audiometry, allow a variety of middle ear and other disorders to be diagnosed.

The ear probe preferably comprises a conventional ear probe tip that is preferably configured for fitting with an ear tip for insertion into an ear canal of the patient and for sealing the ear canal with an air-tight seal.

The ear probe tip may provide an output of an air conduit connected with the air conduit of the cable to the audiologic test apparatus.

The ear tip may be disposable and for example made of rubber.

Screening tests may include tympanometry, Ipsi and Contra acoustic reflex, Ipsi and Contra fast acoustic reflex threshold, pure tone audiometry, speech audiometry, etc.

Clinical tests may include tympanometry, Eustachian tube function, Ipsi and Contra manual acoustic reflex, Ipsi and Contra reflex decay, acoustic reflex latency, pure tone audiometry, speech audiometry, Gene's test, etc.

Preferably, the audiologic test apparatus is configured for performing tests involving measurement and/or emission of sound.

The results of the ear measurements performed with the audiologic test apparatus and the ear probe are recorded by the audiologic test apparatus and stored in a memory of the audiologic test apparatus. The results are stored together with an indication of whether the right ear or the left ear was subject to the testing as selected with the remote user interface.

A remote user interface for connection with an audiologic test apparatus for audiologic testing of a patient via an ear probe, the patient having a right ear and a left ear, the remote user interface includes: a remote user interface housing accommodating a mechanically non-latching switch for selection of one of the right ear and the left ear of the patient for testing with the audiologic test apparatus; wherein the remote user interface housing is configured for mechanical connection with the ear probe of the audiologic test apparatus in such a way that the remote user interface will be located relatively more proximate to the patient than to the audiologic test apparatus during the audiologic testing of the patient.

Optionally, the mechanically non-latching switch is a touch sensitive switch.

Optionally, the touch sensitive switch is positioned so that a surface of the touch sensitive switch is flush with a surface of the remote user interface housing.

Optionally, the mechanically non-latching switch is a push button with tactile feedback.

Optionally, the mechanically non-latching switch is configured for alternating selection of the right ear and the left ear for testing with the audiologic test apparatus.

Optionally, the remote user interface housing is configured to accommodate two mechanically non-latching switches configured for selection of the right ear and left ear, respectively, for testing with the audiologic test apparatus.

Optionally, the remote user interface housing accommodates an indicator for indication of the one of the right ear and the left ear that is selected for testing with the audiologic test apparatus.

Optionally, the indicator is configured to provide visual indication of the one of the right ear and the left ear selected for testing with the audiologic test apparatus.

Optionally, the indicator comprises a light source configured to emit light of a first colour when one of the right ear and the left ear is selected for testing with the audiologic test apparatus, and to emit light of a second colour different from the first colour when the other one of the right ear and the left ear is selected for testing with the audiologic test apparatus.

Optionally, the indicator comprises a sound source configured to emit a specific sound when one of the right ear and the left ear is selected for testing with the audiologic test apparatus.

Optionally, the remote user interface further includes a first connector for connection to the audiologic test apparatus with a first cable, and a second connector for connection to the ear probe with a second cable, so that the ear probe may be connected with the audiologic test apparatus via the remote user interface housing.

Optionally, the remote user interface housing constitutes at least a part of a housing of the ear probe.

An audiologic test apparatus for connection with the remote user interface described herein, the audiologic test apparatus includes a test apparatus housing different from the remote user interface housing, wherein the test apparatus housing accommodates a user interface for selection of the right ear or the left ear of the patient for testing with the audiologic test apparatus.

Optionally, the housing of the audiologic test apparatus accommodates an indicator for indication of the one of the right ear and the left ear that is selected for testing with the audiologic test apparatus.

Optionally, the audiologic test apparatus further includes a non-transitory medium for storing test results of the audiologic test of the selected one of the right ear and the left ear, together with an identification of the selected one of the right ear and the left ear.

Optionally, the audiologic test apparatus further includes the ear probe.

Other features, advantageous, and embodiments will be described in the detailed description.

### BRIEF DESCRIPTION OF THE DRAWINGS

The drawings illustrate the design and utility of embodiments, in which similar elements are referred to by common reference numerals. These drawings may or may not be drawn to scale. In order to better appreciate how the above-recited and other advantages and objects are obtained, a more particular description of the embodiments will be rendered, which are illustrated in the accompanying drawings. These drawings depict only exemplary embodiments and are not therefore to be considered limiting in the scope of the claims.

In the drawings:

FIG. 1 schematically shows a perspective view of a hand-held ear probe with a remote user interface for an audiologic apparatus utilized for testing an ear of a human, and

FIG. 2 schematically shows a perspective view of a remote user interface housing for remote control of an audiologic apparatus.

### DETAILED DESCRIPTION OF THE DRAWINGS

Various illustrative examples of the new remote user interface according to the appended claims will now be described more fully hereinafter with reference to the accompanying drawings, in which various embodiments of new remote user interface are illustrated. The new remote user interface according to the appended claims may, however, be embodied in different forms and should not be construed as limited to the embodiments set forth herein. In addition, an illustrated embodiment needs not have all the aspects or advantages shown. An aspect or an advantage described in conjunction with a particular embodiment is not necessarily limited to that embodiment and can be practiced in any other examples even if not so illustrated, or if not so explicitly described.

As used herein, the singular forms “a,” “an,” and “the” refer to one or more than one, unless the context clearly dictates otherwise.

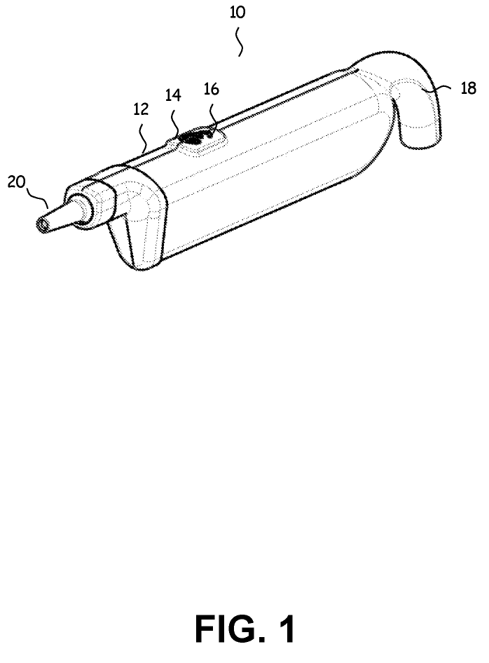

FIG. 1 shows schematically and in perspective a hand-held ear probe 10 with a probe housing 12 and configured for connection with an audiologic apparatus (not shown) and comprising a remote user interface 14 for operation of the audiologic apparatus. The remote user interface 14 comprises a mechanically non-latching switch 16 for selection of the right ear or the left ear of the patient for testing with the ear probe and the audiologic apparatus.

In this way, the operator of the ear probe need not move to the audiologic test apparatus during testing for selection of the right ear or the left ear for testing with the audiologic test apparatus. The mechanically non-latching switch may be a push button with appropriate tactile feedback, when the mechanically non-latching switch is actuated, i.e. depressed, in order to perform a selection of the right or left ear.

The illustrated mechanically non-latching switch 16 is configured for alternate selection of the right ear or the left ear for testing by repeated actuation of the mechanically non-latching switch. The mechanically non-latching switch 16 may be a push button, e.g. with appropriate tactile feedback, when the mechanically non-latching switch 16 is actuated, i.e. depressed, in order to perform a selection of the right or left ear.

In another embodiment (not shown), the mechanically non-latching switch 16 may be a touch sensitive switch, e.g. forming part of the surface of the probe housing 12, and preferably the surface of the touch sensitive switch is flush with the surrounding surface of the probe housing 12.

In another embodiment (not shown), the probe housing 12 accommodates two mechanically non-latching switches, one for selection of the right ear and one for selection of the left ear, respectively.

In another embodiment (not shown), the probe housing 12 also accommodates a mechanically non-latching switch configured for controlling start and stop of testing with the audiologic apparatus.

The illustrated mechanically non-latching switch 16 comprises a lamp (not visible) that illuminates the mechanically non-latching switch from inside the mechanically non-latching switch with light of different colours in response to the actual selection of the ear to be tested so that the illustrated mechanically non-latching switch 16 also constitutes an indicator of the one of the right ear or left ear that is currently selected for testing.

The lamp of the illustrated mechanically non-latching switch 16 emits orange light when the right ear is selected for testing and emits blue light when the left ear is selected for testing.

In another embodiment (not shown), the indicator may comprise a sound source, such as a miniature loudspeaker, instead of a light source, or in addition to the light source, wherein the sound source emits a specific sound when a respective one of the right ear and left ear is selected for testing and that emits a different sound when the other one of the right ear and left ear is selected for testing. The two different sounds may be pure tones of different frequencies, or short different melodies, or different sequences of the same sound, e.g. one beep when the right ear is selected for testing and two beeps when the left ear is selected for testing.

In another embodiment (not shown), the probe housing 12 accommodates a lamp configured to indicate measurement status, such as measurement is in progress, air leakage, instability of the measured ear cavity volume, etc.

During testing with the audiologic test apparatus (not shown), the ear probe 10 is connected with a cable 18 to the audiologic test apparatus in a well-known way. The cable 18 includes electrical conductors (not visible) for interconnection of a microphone (not visible) and a loudspeaker (not visible) and the mechanically non-latching switch and indicator 16 accommodated in the probe housing 12. The cable also 18 has an air conduit (not visible) for interconnection with a pump (not shown) in the audiologic test apparatus (not shown) for control of the air pressure in the ear canal subjected to clinical testing or screening in a way well-known in the art of audiologic testing.

Typically, the hand-held ear probe 10 is used for acoustic impedance and static pressure measurements in the ear canal. These measurements, possibly in combination with pure tone audiometry or speech audiometry, allow a variety of middle ear and other disorders to be diagnosed.

The ear probe 10 comprises a conventional ear probe tip 20 preferably configured for fitting with an ear tip (not shown) for insertion into an ear canal of the patient and for sealing the ear canal with an air-tight seal. The probe tip 20 is connected with the air conduit of the cable 18 and the pump (not shown) of the audiologic test apparatus (not shown).

Screening tests may include tympanometry, Ipsi and Contra acoustic reflex, Ipsi and Contra fast acoustic reflex threshold, pure tone audiometry, speech audiometry, etc.

Clinical tests may include tympanometry, Eustachian tube function, Ipsi and Contra manual acoustic reflex, Ipsi and Contra reflex decay, acoustic reflex latency, pure tone audiometry, speech audiometry, Gene's test, etc.

The results of the ear measurements performed with audiologic test apparatus and the ear probe are recorded by the audiologic test apparatus and stored in a memory of the audiologic test apparatus. The results are stored together with an indication of whether the right ear or the left ear was subject to the testing as selected with the remote user interface.

When clinical or screening tests are performed, there is a need for recording which of the left ear or the right ear that is subjected to the test with the ear probe.

For this purpose, the mechanically non-latching switch 16 is conveniently provided at the hand-held ear probe 10 for selection of which of the left ear or the right ear that is to be subjected to the next audiologic test. For example, a reflex test may be performed, wherein the left ear is selected to be tested as ipsi and these selections may be displayed on a screen of the audiologic test apparatus as “Reflex: Left”.

In this way, the operator of the ear probe 10 may perform selection of the ear to be subjected to the next test without having to move proximate to the audiologic test apparatus connected with the ear probe for the purpose of ear selection.

For example, when operating a hand held ear probe, the operator may have established an air-tight seal of the ear canal with the hand held ear probe before recognizing the need to select the ear in question before the test can be performed. Conventionally, the operator then had to remove the hand held ear probe from the ear to be tested in order to go to the audiologic test apparatus to perform the selection of the right or left ear and revert to the patient and re-establish the seal. This is avoided with the new hand-held ear probe 10 with its remote user interface 14.

Thus, cumbersome and time consuming operator movement is avoided between the audiologic test apparatus and the patient in order to select which ear that is going to be tested and in order to select the other ear when testing of the first ear has been performed. Time is saved and communication with the patient is maintained and disturbances are minimized during audiologic testing with the hand-held ear probe 10.

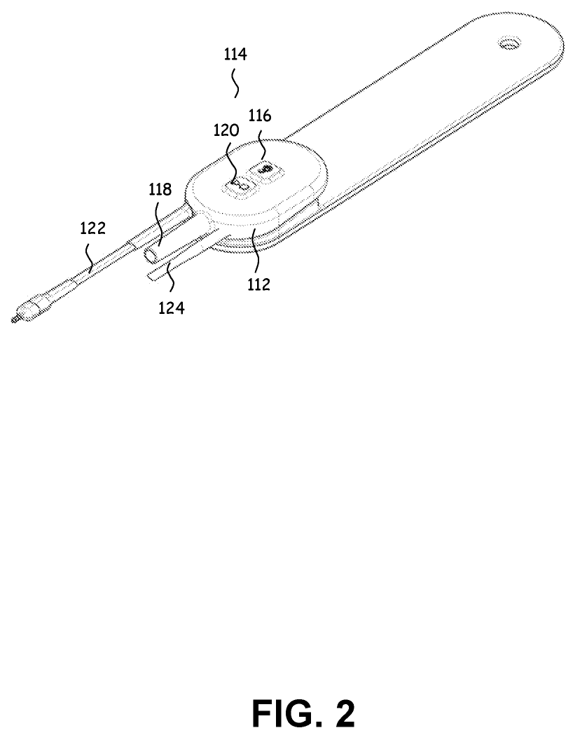

FIG. 2 shows schematically and in perspective a remote user interface housing 112 that accommodates the remote user interface 114 and is intended to rest at the patient's body. The remote user interface 114 is connected to the audiologic test apparatus (not shown) through port 118 with a cable (not shown) that may rest together with the remote user interface housing 112 across a shoulder (not shown) of the patient.

The ear probe (not shown) to be inserted in the selected ear may be connected with a cable 124 (partly shown) to the remote user interface housing 112 in a well-known way. The ear probe cable 124 includes electrical conductors for interconnection of a microphone and a loudspeaker and switches and possible indicators, respectively, accommodated in the probe housing. The ear probe cable 124 also has an air conduit for interconnection with a pump in the audiologic test apparatus for control of the air pressure in the ear canal subjected to clinical testing or screening in a way well-known in the art of audiologic testing.

When required, a second ear probe (not shown) to be inserted into the other ear than the selected ear may be connected with cable 122 to the remote user interface housing 112 in a well-known way. The cable 112 may be similar to the cable 124.

The remote user interface housing 112 assists in maintaining the various cables 122, 124 in convenient and appropriate locations during performance of the clinical or screening tests.

The remote user interface 114 operates with the same means and in the same way as the remote user interface 14 explained above in connection with FIG. 1.

In addition to the advantages of the remote user interface mentioned above with relation to FIG. 1, provision of the remote user interface 114 in a separate remote user interface housing 112 leads to the advantage that no force is applied to the ear probe when the remote user interface 114 accommodated in the remote user interface housing 112 is used, e.g. when the mechanically non-latching switch 116 for ear selection is depressed and released, thereby avoiding that the ear probe is inadvertently moved away from a desired position, e.g. breaking a previously established air tight seal.

The illustrated remote user interface 114 also comprises a mechanically non-latching switch 120 configured for controlling start and stop of testing with the audiologic apparatus in a way well-known in the art.

Although particular embodiments have been shown and described, it will be understood that it is not intended to limit the claimed inventions to the preferred embodiments, and it will be obvious to those skilled in the art that various changes and modifications may be made without department from the spirit and scope of the claimed inventions. The specification and drawings are, accordingly, to be regarded in an illustrative rather than restrictive sense. The claimed inventions are intended to cover alternatives, modifications, and equivalents.

1. A remote user interface for connection with an audiologic test apparatus for audiologic testing of a patient via an ear probe, the patient having a right ear and a left ear, the remote user interface comprising:
a remote user interface housing accommodating a mechanically non-latching switch for selection of one of the right ear and the left ear of the patient for testing with the audiologic test apparatus;
wherein the remote user interface housing is configured for mechanical connection with the ear probe of the audiologic test apparatus in such a way that the remote user interface will be located relatively more proximate to the patient than to the audiologic test apparatus during the audiologic testing of the patient.

2. The remote user interface according to claim 1, wherein the mechanically non-latching switch is a touch sensitive switch.

3. The remote user interface according to claim 2, wherein the touch sensitive switch is positioned so that a surface of the touch sensitive switch is flush with a surface of the remote user interface housing.

4. The remote user interface according to claim 1, wherein the mechanically non-latching switch is a push button with tactile feedback.

5. The remote user interface according to claim 1, wherein the mechanically non-latching switch is configured for alternating selection of the right ear and the left ear for testing with the audiologic test apparatus.

6. The remote user interface according to claim 1, wherein the remote user interface housing is configured to accommodate two mechanically non-latching switches configured for selection of the right ear and left ear, respectively, for testing with the audiologic test apparatus.

7. The remote user interface according to claim 1, wherein the remote user interface housing accommodates an indicator for indication of the one of the right ear and the left ear that is selected for testing with the audiologic test apparatus.

8. The remote user interface according to claim 7, wherein the indicator is configured to provide visual indication of the one of the right ear and the left ear selected for testing with the audiologic test apparatus.

9. The remote user interface according to claim 8, wherein the indicator comprises a light source configured to emit light of a first colour when one of the right ear and the left ear is selected for testing with the audiologic test apparatus, and to emit light of a second colour different from the first colour when the other one of the right ear and the left ear is selected for testing with the audiologic test apparatus.

10. The remote user interface according to claim 7, wherein the indicator comprises a sound source configured to emit a specific sound when one of the right ear and the left ear is selected for testing with the audiologic test apparatus.

11. The remote user interface according to claim 1, further comprising a first connector for connection to the audiologic test apparatus with a first cable, and a second connector for connection to the ear probe with a second cable, so that the ear probe may be connected with the audiologic test apparatus via the remote user interface housing.

12. The remote user interface according to claim 1, wherein the remote user interface housing constitutes at least a part of a housing of the ear probe.

13. An audiologic test apparatus for connection with the remote user interface of claim 1, the audiologic test apparatus comprising a test apparatus housing different from the remote user interface housing, wherein the test apparatus housing accommodates a user interface for selection of the right ear or the left ear of the patient for testing with the audiologic test apparatus.

14. The audiologic test apparatus according to claim 13, wherein the housing of the audiologic test apparatus accommodates an indicator for indication of the one of the right ear and the left ear that is selected for testing with the audiologic test apparatus.

15. The audiologic test apparatus according to claim 13, further comprising a non-transitory medium for storing test results of the audiologic test of the selected one of the right ear and the left ear, together with an identification of the selected one of the right ear and the left ear.

16. The audiologic test apparatus of claim 13, further comprising the ear probe.
